(19) United States
(12) Patent Application Publication
(10) Pub. No.: US 2018/0280517 A1
(43) Pub. Date: Oct. 4, 2018

(54) Nanocomposite, a Preparation Method Thereof and Method for Treating Cancer Using the Same

(71) Applicant: National Chiao Tung University, Hsinchu City (TW)

(72) Inventors: Wei-Ting Huang, Taitung City (TW); Yu-Chien Chiang, Kaohsiung City (TW); Dean-Mo Liu, Zhubei City (TW)

(73) Assignee: National Chiao Tung University

(21) Appl. No.: 15/710,429

(22) Filed: Sep. 20, 2017

(30) Foreign Application Priority Data
Mar. 29, 2017 (TW) 106110485

(51) Int. Cl. A61K 47/36; C07K 16/28; A61K 39/395; A61K 47/16

(52) U.S. Cl. CPC A61K 47/36; C07K 16/2803; A61K 31/7068; A61K 47/16; A61K 39/39558

(57) ABSTRACT

A nanocomposite, a preparation method thereof and a method for treating cancer using the same are provided. The preparation method includes: mixing a first solution including an amphiphilic chitosan and a second solution including anti-cancer components, wherein the anti-cancer components includes gemcitabine, curcumin, the derivatives and combinations thereof; forming a nanoparticle encapsulating the anti-cancer component by a self-assembling process of the amphiphilic chitosan, and binding the nanoparticle with a targeting molecule having specificity to a cancer so as to obtain a nanocomposite. When dissolving the nanocomposite of the present invention after drying into water phase, the nanocomposite still has the same morphology and characteristic before it is dried, so it is convenient for storage and delivery. Additionally, the preferable ratio of gemcitabine and demethoxycurcumin will bring a synergistic effect on cancer therapy.

### CROSS-REFERENCE TO RELATED APPLICATION

This application claims priority from Taiwan Patent Application No. 106110485, filed on Mar. 29, 2017 at the Taiwan Intellectual Property Office, the content of which is hereby incorporated by reference in its entirety for all purposes.

### BACKGROUND OF THE INVENTION

### 1. Field of the Invention

The present relates to a nanocomposite, a preparation method thereof and a method for treating cancer using the same. The present invention specifically provides a cancer specific nanocomposite including medical components having a preferable ratio, a preparation method thereof and a method for treating cancer using the nanocomposite.

### 2. Description of the Related Art

Cancer, or malignant tumor, is a group of diseases involving abnormal cell growth in the body. Rate of cancer is worldwide increasing because of lifestyle changes, increases in risk of radiation exposure, and more and more environmental oncogenic factors. In 2012, approximately 14,100,000 were diagnosed with cancer, in which nearly 8,200,000 died. Cancer accounted for approximately 14.6% of deaths globally according to World Cancer Report 2014. Effective cancer treatments are therefore still urgently demanded even within the world with advanced medical technology.

The treatments of cancer include surgical resection, chemotherapy, radiotherapy, monoclonal antibody therapy, targeted therapy, etc., wherein the targeted therapy is considered to be a more effective and less harmful treatment and is popular in this section. There are two main medicaments used in targeted therapies, specific small molecules and nanoparticles including specific molecules. Since the nanoparticle has a property of carrying an amount of anti-cancer components, it is more effective than small molecule alone in cancer treatment. Additionally, the nanoparticle can release anti-cancer components in specific locations, which make its cancer treating effect comparable to chemotherapy but with less harmful side effects.

Despite the advantages of the nanoparticle aforementioned, long term preservation or storage is still an issue. Nanoparticles are usually preserved in a form of colloidal solution (Taiwan patent No. I458833, I482782, I399214, etc.). In order to avoid aggregation, protection agents are added to the surface of modified nanoparticles or in the nanoparticle solution. Nanoparticle colloidal solution is also sensitive to temperature variation, which make it harder to preserve and transport. Nanoparticles can also be dried and preserved in a form of nanopowder and then redissolved into water phase for being administered to a patient. The problem is, after dissolved, the nanoparticles are no long be brought back to their previous particle size because of aggregation phenomenon, which degrades the effect of the medication.

Lately, the study of multiple drugs combination shows promising therapeutic effects against cancers. For example, FDA approved erlotinib in combination with gemcitabine for the treatment of advanced pancreatic cancer in 2005; the combination treatment of irinotecan and docetaxel and the combination treatment of bevacizumab and cetuximab were disclosed at ASCO Annual Meeting in 2007; phase III trials of erlotinib and gemcitabine combination for non-small cell lung cancer were shown in the paper in 2012 (DOI: 10.1200/JCO.2011.39.9782 Journal of Clinical Oncology 30, no. 28 (October 2012) 3516-3524). Although showing benefits, five-year survival rate still does not improve in clinical.

In conclusion, there are demands of finding a drugs combination ratio for specific cancers, target nanoparticle medicament capable of long-term preservation and transportation, or their combination.

### SUMMARY OF THE INVENTION

A purpose of the present invention is to solve the aforementioned problems by providing a nanocomposite, a preparation method and a method for treating cancer using the same.

The preparation method of the nanocomposite includes the steps of: mixing a first solution including an amphiphilic chitosan with a second solution including one or a plurality of anti-cancer components, wherein the anti-cancer components includes gemcitabine, curcumin, their derivatives, or any combination thereof; forming a nanoparticle encapsulating one or a plurality of anti-cancer components by a self-assembling process of the amphiphilic chitosan; modifying the nanoparticle by binding a targeting molecule having specificity to a cancer to form the nanocomposite.

Preferably, the amphiphilic chitosan includes a hydrophilic end comprising gadolinium.

Preferably, the first solution includes the amphiphilic chitosan at a concentration of about 0.001% (w/w) to 10% (w/w).

Preferably, the second solution includes one or a plurality of anti-cancer components at a concentration of about 1 mg/mL to 1000 mg/mL

Preferably, the second solution includes dimethyl sulfoxide or alcohol.

Preferably, the ratio by weight between the curcumin and its derivatives and the gemcitabine and its derivatives is about 1:1 to 1:60.

Preferably, the targeting molecule includes an EGFR antibody, a CD-133 antibody, a CD-166 antibody, or a PD-L1 antibody.

Preferably, the targeting molecule is bound to the nanoparticle through a crosslinking agent.

Preferably, the crosslinking agent includes 3-(ethyliminomethyleneamino)-N,N-dimethyl-propan-1-amine (EDC, EDAC, or EDCI)

Another purpose of the present invention is to provide the nanocomposite manufactured by the aforementioned method.

Preferably, when the nanocomposite is dissolved in a solvent, the nanocomposite has a particle size about 5 nm to 500 nm.

Preferably, a solvent of the nanocomposite can be removed through a process including freeze-drying, vacuum concentration, vacuum drying, spray drying, or any combination thereof to form a dried micron powder having a particle size about 0.5 μm to 20 μm.

Preferably, when the micron powder is dissolved in a solvent, the micron powder is dispersed into the nanocomposite, the particle size of which is about 5 nm to 500 nm.

Preferably, the nanocomposite is in a form of solution ampoule, oral tablet, or inhalant for administrating.

Another purpose of the present invention is to provide a method for treating cancer using the nanocomposite prepared by the aforementioned method.

Preferably, the cancer includes non-small cell lung cancer, small cell lung cancer, ovarian cancer, pancreatic carinoma, bladder cancer, breast cancer, or brain cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The figures are used to illustrate the embodiments for one skilled in the art to comprehend the present invention but not to limit the present invention.

FIG. 1 is a schematic diagram showing a preparation procedure of a nanocomposite of the present invention.

FIG. 2 shows a TEM image of a nanocomposite.

FIG. 3 shows graphs of gemcitabine (GEM) release percentage versus time.

FIG. 4 shows graphs of demethoxycurcumin (DMC) release percentage versus time.

FIG. 5 is a graph of drug combination index with fraction affected (Fa) calculated based on the viability of A549-ON cell lines.

FIG. 6 shows the sizes of tumors versus days after being respectively administered with different kinds of medicaments.

FIG. 7 is a graph of CI versus Fa calculated based on the viability of A549 cell lines.

FIG. 8 shows the size of A549 ectopic tumor versus days after being respectively treated with different kinds of medicaments. The arrows indicate time points of the treatments.

FIG. 9 shows a comparative diagram of inhibition efficiency of A549 ectopic tumor.

FIG. 10 shows images including: (A) an image showing a dried micron powder of the present invention; (B) an image showing particle morphology of a dried micron powder; and (C) an image showing particle morphology of a dried micron powder after dissolved in a solvent.

### DESCRIPTION OF THE PREFERRED EMBODIMENTS

The purpose of the herein described embodiments is to illustrate the technical ideas and features of the present invention, such that one skilled in the art can comprehend the contents of the present invention and practice said invention accordingly.

The expressions and acronyms and the meanings thereof used in this invention are listed in Table 1.

TABLE 1

| Abbreviation | Meaning |
|---|---|
| DMC | Demethoxycurcumin |
| GEM | Gemcitabine |
| CHC | Amphiphilic chitosan |
| CHC/GEM | CHC nanoparticle encapsulating GEM |
| CHC/DMC | CHC nanoparticle encapsulating DMC |
| CHC/DMC-GEM | CHC nanoparticle encapsulating GEM |
| | and DMC |
| CHC/DMC-GEM/anti-CD133 | CHC/GEM-DMC binding with anti- |
| | CD133 |
| CHC/DMC-GEM/anti-EGFR | CHC/GEM-DMC binding with anti-EGFR |
| CHC/anti-EGFR | CHC nanoparticle binding with anti- |
| | EGFR |
| CI | Combination index |
| Fa | Fraction affected |
| A549 | Non-small cell lung cancer cell line |
| A549-ON | Non-small cell lung cancer stem cell line |
| IC₅₀ | Half maximal inhibitory concentration |
| ED₅₀ | Median effective dose |
| PBS | Phosphate-buffered saline |
| EGFR | Epidermal growth factor receptor |

Amphiphilic chitosan or CHC in this invention means chemically modified chitosan such that the resulted component has a hydrophobic group, an original hydrophilic end, and a functionally modified hydrophilic end. Therefore, the modified chitosan includes both hydrophilic end and hydrophobic end.

The expression of “encapsulating” in the present invention means an additional substance is carried in an internal space of a nanoparticle. For example, a CHC nanoparticle encapsulating GEM means GEM is carried in an internal space of a CHC nanoparticle.

The expression of “release” in the present invention means an encapsulated component is freed from the encapsulating nanoparticle. During releasing, the nanoparticle may either be broken or not.

The combination index or CI means a value acquired through a calculation based on the Combination Index Theorem. From CI value, the interaction between components within a drug having multiple components can be understood. For example, the CI value quantitatively defines synergism (CI<1), additive effect (CI=1), and antagonism (CI>1) among components.

The expression fraction affected (Fa) means the fraction affected of a component according to Median-Effect Principle. A CI-Fa plot can be used to define the synergism and antagonism relation between different components.

The ratio between DMC and GEM in the present invention is weight ratio.

In an aspect of the present invention, the nanocomposite is prepared by one-pot synthesis as shown in FIG. 1.

In one embodiment, before one-pot synthesis, a first solution is prepared by adding the amphiphilic chitosan powder into double distilled water, wherein the concentration of the amphiphilic chitosan in the first solution is about 0.001% to 10% (w/w, the ratio of the weight of the amphiphilic chitosan to the weight of the first solution), or preferably about 0.005% to 7.5% (w/w), or preferably about 0.01% to 5% (w/w), or preferably about 0.025% to 2.5% (w/w), or more preferably about 0.05% (w/w).

In one embodiment, the anti-cancer component may include gemcitabine, curcumin, gemcitabine derivative(s), curcumin derivative(s), or any combination thereof. Preferably, the anti-cancer component includes gemcitabine (GEM) and demethoxycurcumin (DMC). In one embodiment, demethoxycurcumin powder is first mixed with gemcitabine powder with a ratio between 1:1 to 1:500, or preferably 1:5, or preferably 1:10, or preferably 1:20, or preferably 1:25, or preferably 1:50, or preferably 1:100, or preferably 1:150, or preferably 1:200, and the mixture is then dissolved in dimethyl sulfoxide or alcohol to form a second solution, wherein the concentration of the overall anti-cancer component in the second solution is about 1 mg/mL to 1000 mg/mL, or preferably 100 mg/mL to 900 mg/mL, or preferably 300 mg/mL to 700 mg/mL, or preferably 400 mg/mL to 600 mg/mL. In a preferable embodiment, GEM and DMC are dissolved in dimethyl sulfoxide or in alcohol.

In one embodiment, the first solution with 0.05% (w/w) amphiphilic chitosan is mixed with the second solution having demethoxycurcumin to gemcitabine ratio equal to 1:5, and the mixed solution is then stirred at 4° C. for 24 hours to form CHC/DMC-GEM. In one embodiment, CHC/DMC-GEM is mixed with crosslinking agent and targeting molecule to bind the targeting molecule to the CHC/DMC-GEM to form CHC/DMC-GEM/targeting molecule, wherein the crosslinking agent is preferably 3-(ethyliminomethyleneamino)-N,N-dimethyl-propan-1-amine (EDC) and the targeting molecule is preferably anti-EGFR, anti-CD133, anti-CD166, or anti-PD-L1. In a preferable embodiment, CHC/DMC-GEM/targeting molecule may be CHC/DMC-GEM/anti-CD133.

In an embodiment of the present invention, the particle size of the particle in the nanocomposite is about 5 nm to 500 nm, or preferably about 50 nm to 400 nm, or preferably about 100 nm to 250 nm, or more preferably about 150 nm to 200 nm, and the nanocomposite preferably has a negative surface potential.

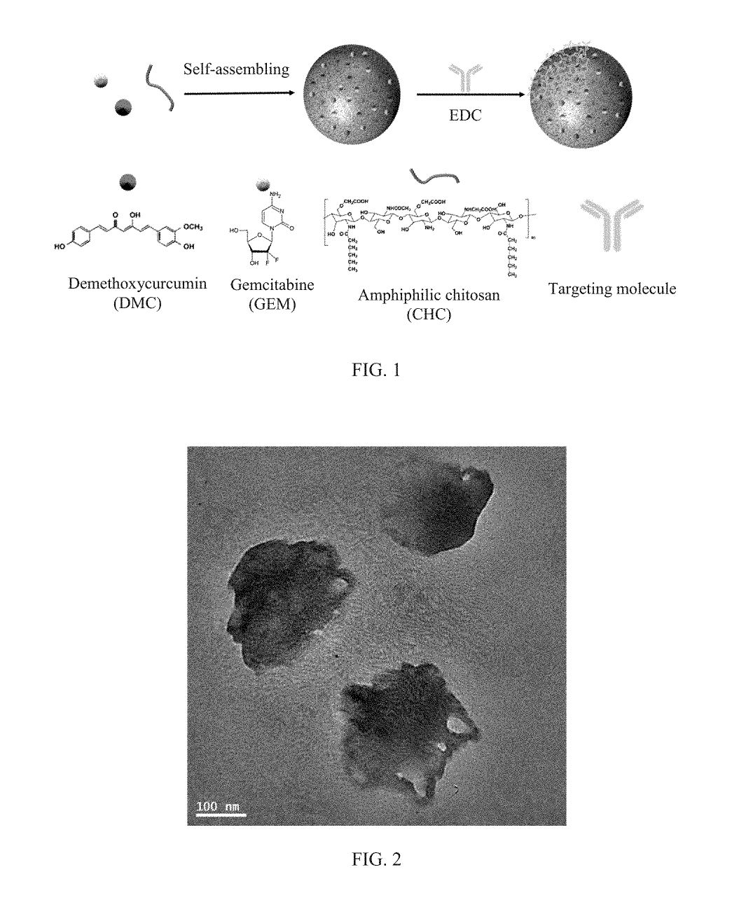

In one embodiment, dynamic light scattering (DLS) is used to measure particle size and surface potential of the CHC/DMC-GEM/targeting molecule, and TEM is used to show its image. In a preferable embodiment, CHC nanoparticle without encapsulating anti-cancer component, CHC/DMC-GEM, and CHC/DMC-GEM/anti-CD133 are respectively measured using DLS by the inventors. The results are shown in Table 2. In a preferable embodiment, an image of CHC/DMC-GEM/anti-CD133 is taken using TEM, as shown in FIG. 2.

TABLE 2
DLS results

| | CHC | | CHC/DMC- |
|---|---|---|---|
| Sample | nanoparticle | CHC/DMC-GEM | GEM/anti-CD133 |
| diameter (nm) | 55 ± 2.1 | 120 ± 4.5 | 180 ± 5.5 |
| surface | −22.5 ± 0.5 | −15.0 ± 3.5 | −6.1 ± 0.3 |
| potential (mV) | | | |

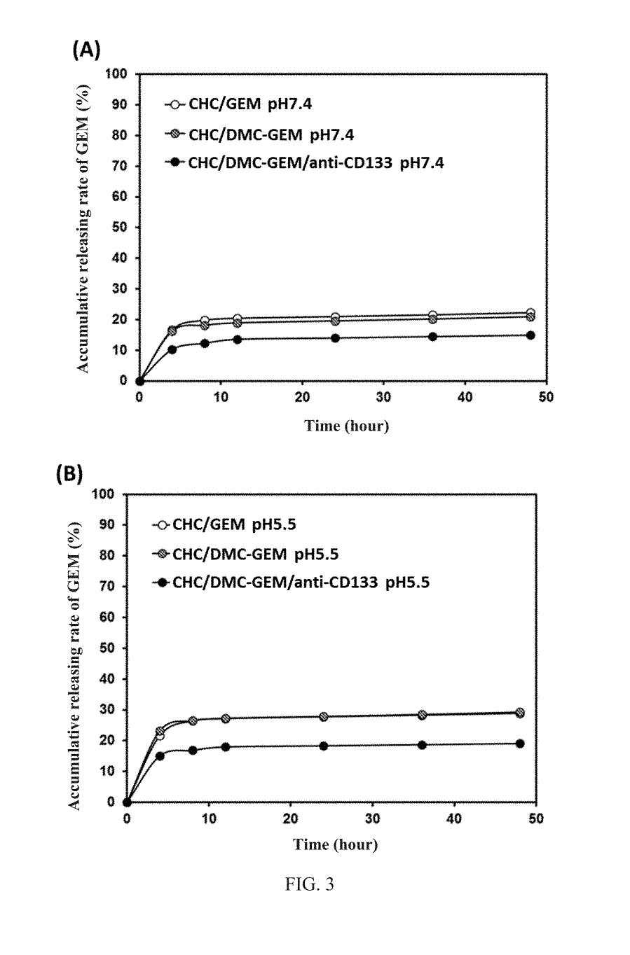

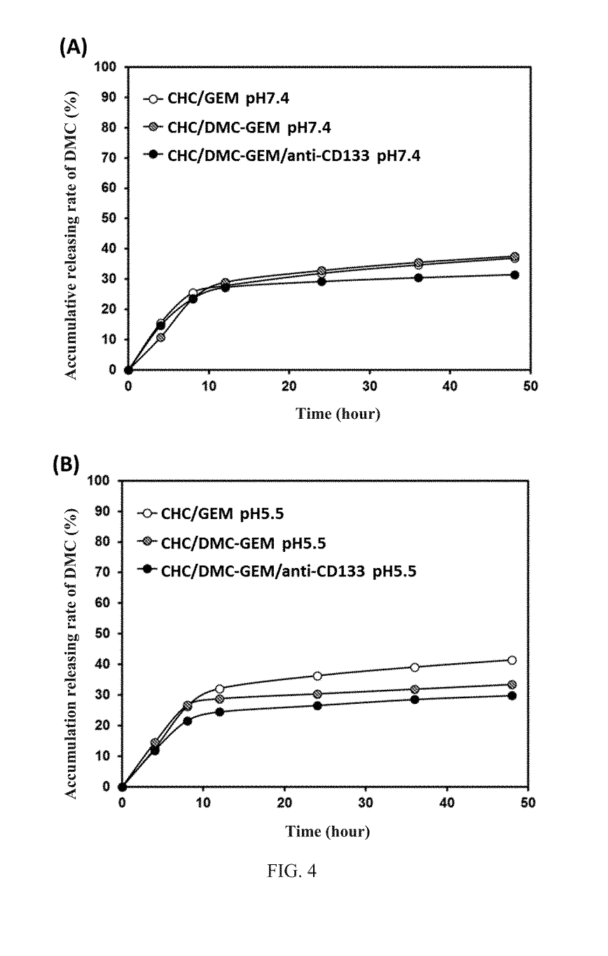

On an embodiment, GEM and DMC releasing rates are examined by the inventors on the samples of CHC/DMC, CHC/GEM, CHC/DMC-GEM, and CHC/DMC-GEM bound with antibody in buffer solutions with various pH values. FIG. 3(A) and FIG. 3(B) show a plot of the GEM accumulation releasing rate versus time, while FIG. 4(A) and FIG. 4(B) show a plot of the DMC accumulation releasing rate versus time of the aforementioned samples, wherein the accumulation releasing rate is the accumulating releasing amount divided by the initially encapsulated amount times 100%. As shown in FIG. 3(A), FIG. 3(B), FIG. 4(A), and FIG. 4(B), the anti-cancer components DMC and GEM has higher releasing rate in the first 10 hours, and the releasing rate becomes slower after. The accumulation releasing rates of these samples are still under 40% after 40 hours. CHC/GEM-DMC/anti-CD133 has the lowest accumulation releasing rate among these samples, which make it a better anti-cancer component carrier form circulating through the body.

In an embodiment of the present invention, the selection of the targeting molecule depends on the type of the cancer of the body, which can include non-small cell lung cancer, small cell lung cancer, ovarian cancer, pancreatic carinoma, bladder cancer, breast cancer, or brain cancer.

In a preferable embodiment, A549-ON is chosen as the cancer cell model. A549-ON is incubated with DMC, GEM, CHC/DMC, and CHC/GEM respectively to observe cell viability. The results are shown in Table 3. In Table 3, DMC and GEM encapsulated by CHC has smaller IC₅₀ comparing to those without CHC encapsulation, providing that an anti-cancer component encapsulated by CHC has higher toxic effect to the cells.

TABLE 3

| Sample | A549-ON, IC₅₀ (μg/mL) |
|---|---|
| DMC | 10 |
| GEM | 116.6 |
| CHC/DMC | 8.37 |
| CHC/GEM | 73.94 |

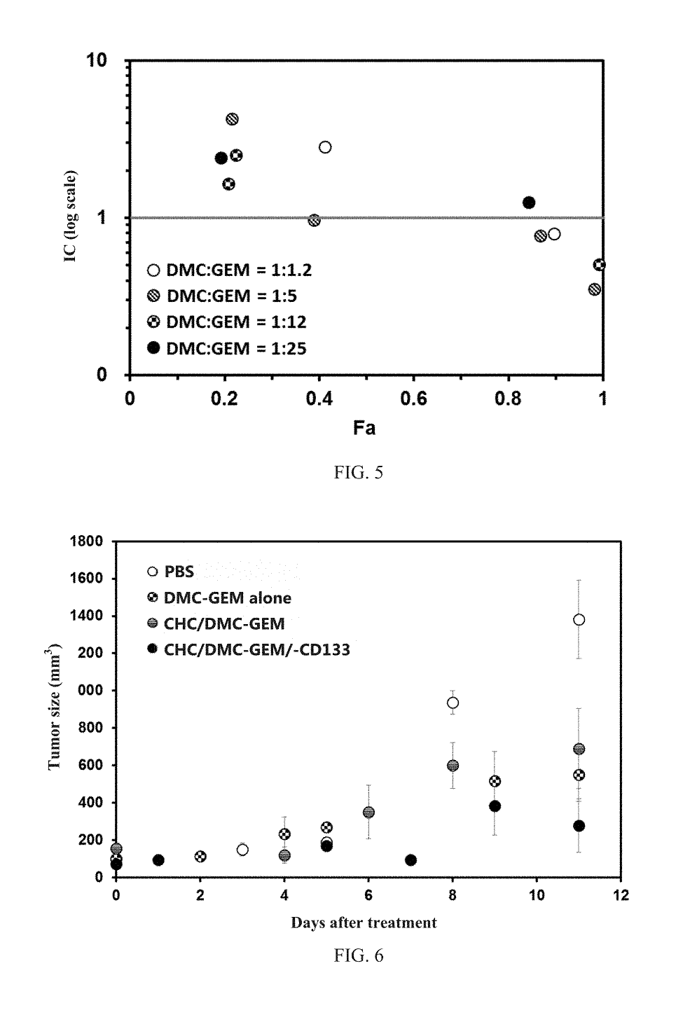

In an embodiment, a plurality of CHC/DMC-GEM having different DMC to GEM ratio including 1:1.2, 1:5, 1:12, and 1:25 are respectively incubated with A549-ON to find a component ratio with better synergistic effect. After calculation, a CI versus Fa plot is shown in FIG. 5 In FIG. 5, CI value is less than 1 when Fa is equal to 0.5 in the condition of CHC/DMC-GEM having DMC to GEM ratio equal to 1:5, i.e. DMC to GEM ratio is equal to 1:5 when preparing CHC/DMC-GEM, DMC and GEM show synergism effect and better treatment result.

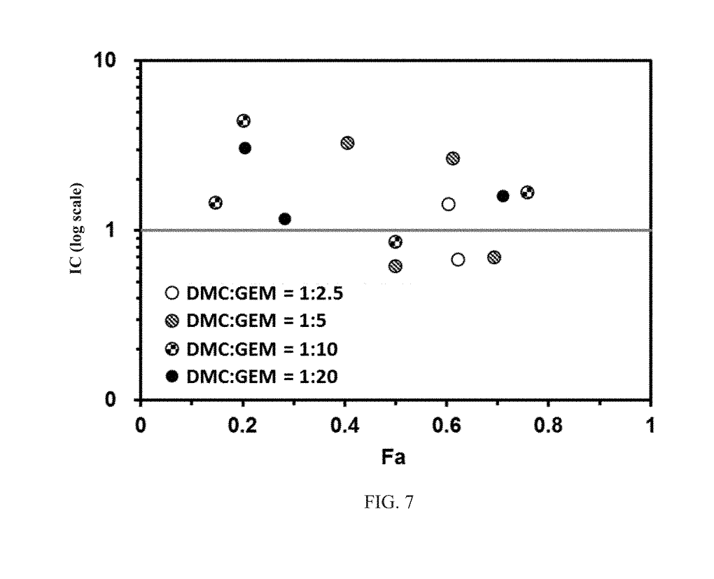

In an embodiment, malignant ectopic tumor A549-ON bearing mice are respectively treated with PBS, a mixture of DMC and GEM, CHC/DMC-GEM of the present invention, and CHC/DMC-GEM/anti-CD133 of the present invention. The sizes of tumors are recorded during 11 days after the treatments. As shown in FIG. 6, the tumor size in the control mice, treated with PBS, is 7 times larger than the mice treated with CHC/DMC-GEM/anti-CD133, and CHC/DMC-GEM/anti-CD133 also shows better malignant A549-ON ectopic tumor inhibition ability than the other substances.

In another preferable embodiment, A549 cells, which are selected as cancer cell model, are respectively incubated with DMC, GEM, CHC/DMC, and CHC/GEM. The viabilities of cells are shown in Table 4. In Table 4, DMC or GEM encapsulated by CHC has smaller IC₅₀ comparing to those without CHC encapsulation, proving that an anti-cancer component encapsulated by CHC has higher toxic effect to the cells.

TABLE 4

| Sample | A549, IC₅₀ (μg/mL) |
|---|---|
| DMC | 10.3 |
| GEM | 116.6 |
| CHC/DMC | 8.37 |
| CHC/GEM | 70.3 |

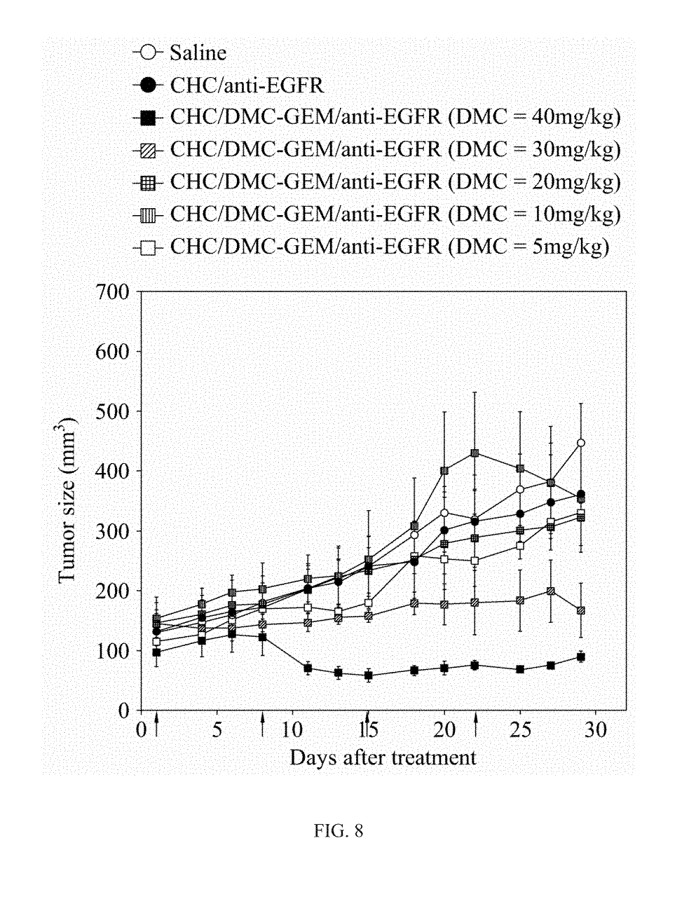

In an embodiment, a plurality of CHC/DMC-GEM having different DMC to GEM ratios including 1:2.5, 1:5, 1:10, and 1:20 are respectively incubated with A549 to find a drug ratio with better synergistic effect. After calculation, a CI versus Fa plot is shown in FIG. 7 based on the viability of A549 cells. When Fa is equal to 0.5, CI value is less than 1 in the condition of CHC/DMC-GEM having DMC to GEM ratio equal to 1:5, which means DMC and GEM in CHC/DMC-GEM show better synergism effect and the treatment may be improved under the condition.

In an embodiment, malignant ectopic tumor A549-ON bearing mice are respectively treated with saline, CHC/anti-EGFR, and CHC/DMC-GEM/anti-EGFR for observing their efficacy in tumor treatment.

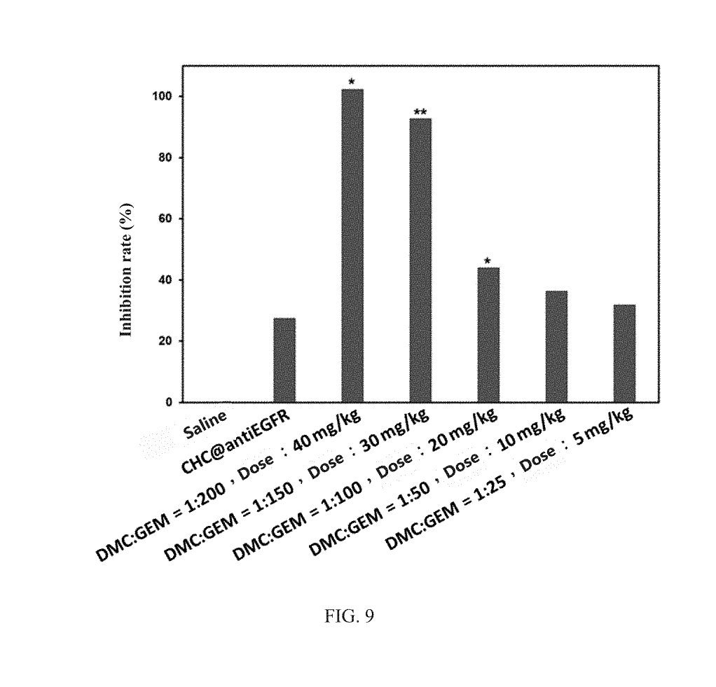

In a preferable embodiment, the ratio of DMC to GEM in CHC/DMC-GEM/anti-EGFR is 1:5, and the DMC doses in CHC/DMC-GEM/anti-EGFR given to the mice are 5 mg/Kg, 10 mg/Kg, 20 mg/Kg, 30 mg/Kg, and 40 mg/Kg respectively. After the first treatment, the same treatment is given to the same mouse on the 8th day, the 15th day, and the 22nd day. During this 29 days experiment, the size of A549 ectopic tumor is recorded. The results are shown in FIG. 8, wherein the arrows indicate when the treatments are given. On the 29th day, subtracting the tumor size of the mouse treated with saline from the tumor sizes of the mice treated with other substances, the tumor inhibition rates can be calculated and presented in a form of column chart. As shown in FIG. 9, the tumor inhibition rates of those treated with CHC/DMC-GEM/anti-EGFR having DMC doses equal to 40 mg/Kg, 30 mg/Kg, and 20 mg/Kg are observable comparing to those treated with saline. As for ED₅₀ of A549 treated with CHC/DMC-GEM/anti-EGFR, calculated GEM is equal to 98.98 mg/Kg and DMC is equal to 19.67 mg/Kg based on the tumor inhibition rates above.

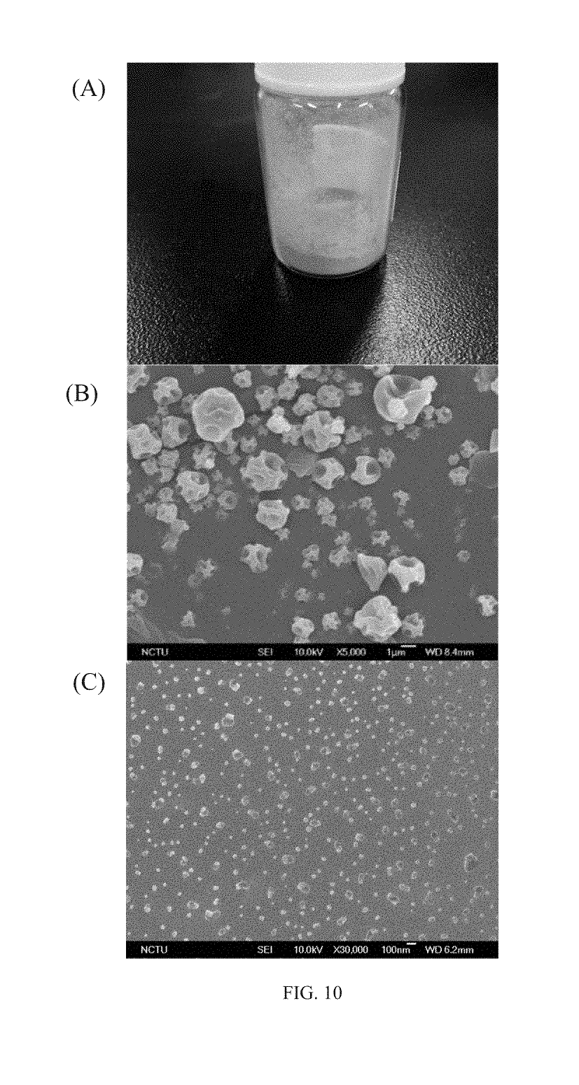

In an embodiment, the solvent of the nanocomposite of the present invention can removed through process including freeze-drying, vacuum concentration, vacuum drying, spray drying, or any combination thereof to form the dried micron powder with particle size about 0.5 μm to 20 μm, or preferably about 0.5 μm to 10 μm, or preferably about 0.5 μm to 5 μm, or more preferably about 0.5 μm to 2 μm. In a preferable embodiment, the dried micron powder can be obtained from the nanocomposite of the present invention through the process of spray granulation. The image of the dried micron powder is shown in FIG. 10(A). The particle morphology and the particle size of the dried micron powder can be seen and inspected by Scanning Electron Microscope. The diameter is about 1 μm. The image is shown in FIG. 10(B). When dissolved in a solvent, water for instance, the dried micron powder is restored back to the form of nanocomposite before drying. The particle size of particles in the nanocomposite is about 100 nm as shown in FIG. 10(C). The image is taken by Scanning Electron Microscope. Therefore, the nanocomposite is not necessary to be preserved in colloidal solution form and can be dried and preserved in powder form for long term storing and transportation. The powder form is also less sensitive to the storage temperature.

In an embodiment, the nanocomposite can be administered in a form of solution ampoule, oral tablet, or inhalant.

In another embodiment, the modified gadolinium on the amphiphilic chitosan included in the nanocomposite of the present invention can also be served as a part of contrast media in T1 MRI.

The foregoing descriptions are merely specific implementation manners of the nanocomposite of the present invention used to develop drugs formulated to treat cancers, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure shall be subject to the protection scope of the claims.

What is claimed is:

1. A preparation method of a nanocomposite, comprising steps of:
mixing a first solution including an amphiphilic chitosan and a second solution including an anti-cancer component;
forming a nanoparticle encapsulating the anti-cancer component by a self-assembling process of the amphiphilic chitosan;
modifying the nanoparticle by binding a targeting molecule having specificity to a cancer on the nanoparticle to form the nanocomposite;
wherein the anti-cancer component includes gemcitabine, curcumin, their derivatives, or a combination thereof.

2. The method of claim 1, wherein the amphiphilic chitosan includes a hydrophilic end comprising gadolinium.

3. The method of claim 1, wherein the concentration of the amphiphilic chitosan in the first solution is about 0.001% (w/w) to 10% (w/w).

4. The method of claim 1, wherein the concentration of the anti-cancer component in the second solution is about 1 mg/mL to 1000 mg/mL.

5. The method of claim 4, wherein the second solution includes a dimethyl sulfoxide or an alcohol.

6. The method of claim 1, wherein the ratio by weight between the curcumin and derivatives thereof and the gemcitabine and derivatives thereof is about 1:1 to 1:60.

7. The method of claim 1, wherein the targeting molecule includes an EGFR antibody, a CD-133 antibody, a CD-166 antibody, or a PD-L1 antibody.

8. The method of claim 1, wherein the targeting molecule is bound to the nanoparticle by a crosslinking agent.

9. The method of claim 8, wherein the crosslinking agent includes 3-(ethyliminomethyleneamino)-N,N-dimethyl-propan-1-amine.

10. A nanocomposite, which is prepared by the method of claim 1.

11. The nanocomposite of claim 10, wherein when the nanocomposite is dissolved in a solvent, the nanocomposite has a particle size about 5 nm to 500 nm.

12. The nanocomposite of claim 10, wherein a solvent of the nanocomposite can be removed through a process including freeze-drying, vacuum concentration, vacuum drying, spray drying, or a combination thereof to form a dried micron powder having a particle size about 0.5 μm to 20 μm.

13. The nanocomposite of claim 12, wherein when the dried micron powder is dissolved in the solvent, the micron powder is dispersed into the nanocomposite, which size is about 5 nm to 500 nm.

14. The nanocomposite of claim 10, wherein the nanocomposite is in a form of solution ampoule, oral tablet, or inhalant for administrating.

15. A method for treating cancer using a nanocomposite prepared by the method of claim 1, comprising:
administrating the nanocomposite to a subject.

16. The method of claim 15, wherein the cancer includes non-small cell lung cancer, small cell lung cancer, ovarian cancer, pancreatic carinoma, bladder cancer, breast cancer, or brain cancer.
